SEQUENCE LISTING (3 sequences)

SEQ ID NO: 1 - RNA, 19 nt, synthetic construct
  source 1..19: mol_type = other RNA
ggaatgcaattctcagaga

SEQ ID NO: 2 - RNA, 19 nt, synthetic construct
  source 1..19: mol_type = other RNA
catgctatggtgttcttca

SEQ ID NO: 3 - protein, 4 aa, synthetic construct
DEVD

(19) United States
(12) Patent Application Publication
(10) Pub. No.: US 2025/0073257 A1
(43) Pub. Date: Mar. 6, 2025

(54) SMALL MOLECULE AND SIRNA INHIBITORS AGAINST LIPOCALIN-2

(71) Applicant: UNIVERSITY OF PUERTO RICO, San Juan, PR (US)

(72) Inventors: Pablo VIVAS-MEJIA, San Juan, PR (US); Eliud HERNANDEZ-O'FARRILL, San Juan, PR (US); Ginette S. SANTIAGO, San Juan, PR (US)

(21) Appl. No.: 18/293,091

(22) PCT Filed: Jul. 27, 2022
(86) PCT No.: PCT/US2022/038570

§ 371 Date: Jan. 29, 2024

Related U.S. Application Data
(60) Provisional application No. 63/227,209, filed on Jul. 29, 2021.

(51) Int. Cl. A61K 31/713; A61K 31/403; A61K 31/427; A61K 31/519; C12N 15/113

(52) U.S. Cl. CPC A61K 31/713; A61K 31/403; A61K 31/427; A61K 31/519; C12N 15/113; C12N 2310/14; C12N 2320/31

(57) ABSTRACT

The present disclosure provides evidence that LCN2 is abundant in IBC cells. and that LCN2 silencing decreases cell proliferation, cell migration, and reduces the invasiveness ability of IBC cells. In vitro targeting of LCN2 with small molecule inhibitors suggests the potential of LCN2 as a plausible target for IBC treatment. Together, these findings support a novel targeted therapeutic approach for an aggressive and deadly invasive cancer, such as IBC.

### CROSS-REFERENCE TO RELATED APPLICATION(S)

This application claims the benefit of priority under 35 U.S.C. § 119 (e) to the U.S. Provisional Patent Application No. 63/227,209, filed Jul. 29, 2021, which is incorporated herein by reference in its entirety.

The claimed invention was made with U.S. Government support under Grant Numbers SC2GM116712, U54CA096297, and U54MD007600, awarded by the National Institutes of Health (NIH). The Government has certain rights in this invention.

### BACKGROUND

Inflammatory Breast Cancer (IBC) is an aggressive locally advanced breast cancer (LABC) subtype that disproportionately affects younger women and has a poor survival outcome. IBC comprises approximately 2-4% of all breast cancer cases in the United States and accounts for 7-10% of all breast cancer-related deaths. The aggressive nature of IBC has been attributed to the high risk of both distant metastasis and locoregional (lymph node and skin) recurrence. Moreover, all molecular subtypes of IBC are more aggressive compared to non-IBC subtypes, having low recurrence-free survival, low overall survival (40% versus 65% for non-IBC patients), and poor therapeutic response.

Current IBC treatment includes a multimodal approach consisting of neoadjuvant chemotherapy (NAT), followed by surgery and postmastectomy radiation therapy. The NAT-therapeutic strategy is based on IBC molecular profiles, including anthracycline-based and/or taxane-based therapy. The incidence of human epidermal growth factor receptor 2 (HER2) and triple-negative breast cancer (TNBC) subtypes are high in IBC. In the case of HER2-positive subtypes, additional HER2-targeted therapy is used. However, the main limitation facing IBC treatment is the lack of specific therapeutic targets.

Lipocalin-2 (LCN2) is a secreted glycoprotein involved in transporting hydrophobic ligands across the cell membrane, modulating the immune response during bacterial infection, and promoting epithelial cell differentiation and iron homeostasis. LCN2 is aberrantly upregulated in cancerous tissues derived from the pancreas, colon, ovaries, and breast. Overexpression of LCN2 is also associated with the progression of aggressive forms of endometrial carcinoma, pancreas, and breast cancers. Particularly, LCN2 is aberrantly abundant in inflammatory breast cancer (IBC) patients, independent of molecular subtype differences. However, the biological consequences of targeting LCN2 using siRNAs or small molecule inhibitors in IBC have not been reported.

### SUMMARY

A target for reducing progression and metastatic of inflammatory breast cancer cells (IBC) is lipocalin-2 (LCN2) a secreted glycoprotein aberrantly abundant in various cancers. The expression of LCN2 in IBC and non-IBC was compared and effects of inhibiting expression of LCN2-calyx by siRNA and small molecules is disclosed. Based on immunoblotting, higher LCN2 protein levels were observed in IBC cells than non-IBC cells. In the latter the LCN2 levels were almost undetectable. The biological effects of targeting LCN2 in IBC cells with small interference RNAs (siRNAs) and small molecule inhibitors are disclosed. siRNA-mediated LCN2 silencing in IBC cells significantly reduced cell proliferation, viability, migration, and invasion. Furthermore, LCN2 silencing promoted apoptosis and arrested the cell cycle progression in the G0/G1 to S phase transition.

In addition, a structure-based virtual screening approach identified potential chemical inhibitors of LCN2. Sixteen (16) potential LNC2 inhibitors were identified in a set of 25,000 ligands from the Asinex library. Molecular docking achieved interactions between residues within the three pockets of the LCN2-calyx and the ligands. Four out of sixteen selected compounds significantly decreased cell proliferation, cell viability and the AKT phosphorylation levels in SUM149 cells. Moreover, ectopically expressing LCN2 MCF7 cells, treated with two LCN2 inhibitors (ZINC00784494 and ZINC00640089) showed a significant decrease in cell proliferation, and the two compounds (ZINC00784494 and ZINC00640089) reduced cell viability and colony formation of IBC cells. LCN2 is a promising target for IBC treatment using siRNA and small molecule inhibitors. Further, LCN2-calyx is a region of the glycoprotein that includes key functional groups of siderophore recognition, and is a suitable target for attack by inhibitors. Siderophores are low molecular weight molecules that chelate with iron.

Aspects of the invention are: 1. A method of reducing progression and metastasis of inflammatory breast cancer cells (IBC) by inhibiting overexpression of lipocalin-2 (LCN2), the method comprising:
(a) obtaining small molecules or siRNAs that selectively target a lipocalin-2 (LCN2)-calyx; and
(b) contacting the IBC cancer cells with at least one of the inhibitors selected from the group consisting of small molecules, siRNAs, or combinations thereof, at concentrations effective to inhibit overexpression of LCN2, as compared to levels of LCN2 in IBC cells not contacted with the inhibitors.

2. The method of claim 1, whereas the small molecules are selected from a group consisting of compounds ZINC00784494, ZINC00640089 and combinations thereof.

3. The method of claim 1, wherein the siRNAs are selected from the group consisting of LCN-2-siRNA1, LCN2-siRNA-2, and combinations thereof.

4. The method of claim 1, wherein IBC cancer cell proliferation and viability are also reduced.

5. The method of claim, 1 further defined as inhibitors combined with standard chemotherapy.

6. The method of claim 1, wherein the IBC cells are from sources wherein neither HER2 nor EGFR are expressed or mutated.

7. The method of claim 3, wherein siRNA mediated LCN2 inhibitors significantly reduced at least one characteristic of IBC cells selected from the group consisting of proliferation, viability, migration, invasion and arrested cell cycle progression in the G0/GI to S phase transition.

8. The method of claim 2, further defined as significantly decreasing cell proliferation, cell viability and AKT phosphorylation levels in SUM 149 cells or equivalents thereof.

9. Compositions comprising nanoliposomal formulations of LCN2-siRNAs.

### BRIEF DESCRIPTION OF THE DRAWINGS

The accompanying drawings, which are included to provide further understanding and are incorporated in and constitute a part of this specification, illustrate disclosed embodiments, and together with the description, serve to explain the principles of the disclosed embodiments. In the drawings:

FIG. 1(A-F): siRNA-based silencing in LCN2-overexpressing IBC cells. (A) Western blot analysis for LCN2 and β-actin (as loading control) in a panel of IBC (SUM149, SUM190, and MDA-IBC3) and non-IBC (MDA-MB-231, SKBR3, and MCF7) cells. (B) Densitometric analysis of band intensities was performed, and values were calculated relative to non-IBC cells, MCF7. Results are shown as Mean±SEM of triplicate experiments, ****P<0.001). Two different siRNAs targeting exon 3 and exon 5 of the human LCN2 sequence (NC_000009.12) were used. Western blot analysis of (C) MDA-IBC3 cells and (D) SUM149 cells were performed after transiently transfected with LCN2-siRNA-1, LCN2-siRNA-2, and negative control-siRNA (NC-siRNA) at 100 nmol/L concentration, as described in the Materials and Methods section. Non treated (NT) cells were treated with the transfection reagent. Densitometric analysis of band intensities of (E) MDA-IBC3 and (F) SUM149 cells was calculated relative to the NC-siRNA. Results are shown as Mean±SEM of triplicate experiments (**P<0.01).

FIG. 2(A-F): LCN2-siRNA-based silencing inhibits colony formation, migration, and invasion of IBC cells. Colony formation assay was performed after LCN2-siRNA-based silencing in MDA-IBC3 and SUM149 cells. Cell proliferation was performed in (A) MDA-IBC3 cells and (B) SUM149 cells. Results are shown as Mean±SEM of triplicate experiments (*P<0.05, **P<0.01, ***P<0.001). (C) Migration assay was performed after LCN2-siRNA transfection (100 nM siRNA, final concentration) in SUM149. (D) NC-siRNA cells represent 100% migration. Images of migrated cells were taken at 20× magnification, scale bar=100 μm. Results are shown as Mean±SEM of triplicate experiments (****P<0.0001). (E) Invasion assay was performed after LCN2-siRNA transfection (100 nM siRNA, final concentration) in SUM149 cells. (F) NC-siRNA cells represent 100% invasion. Images of invaded cells were acquired with a light microscope at 20× magnification., Scale bar=100 μm. Results are shown as Mean±SEM of triplicate experiments (****P<0.0001).

FIG. 3(A-E): LCN2-siRNA-based silencing induces apoptosis and cell cycle arrest in IBC cells. SUM149 cells were transfected with 100 mM of negative control (NC-siRNA) or LCN2 siRNA (siRNA-2). (A) Caspase-3 fluorometric activity assay in SUM149 cells 72 hours after LCN2-siRNA-2 and NC-siRNA transfection. Docetaxel (0.5 nM final concentration) was used as a positive control. (B) Western blot analysis of apoptotic-related proteins. (C) Western blot analysis of cell cycle-related proteins 72 hours after siRNAs transfection. (D, E) Densitometric analysis of the band intensities showed in E. Results are shown as Mean±SEM of triplicate experiments (*P<0.05, ****P<0.0001).

FIG. 4(A-C): Molecular model and docking of ZINC00784494 and ZINC00640089 ligands into LCN2-calyx pocket. (A) Surface model representation of LCN2-calyx pockets. Pockets #1, #2, and #3 (dotted circles) are represented with key amino acid residues in yellow color (right panel). (B) Cartoon docking representation and predicted binding interactions of ligands with key residues of LCN2-calyx pocket. Interactions are represented with yellow dotted lines. Residues are displayed with a three-letter code and numbers representing the position in the polypeptide. (C) Surface docking representation of ligands (represented as sticks) ZINC00784494 (magenta) and ZINC00640089 (yellow) into the LCN2-calyx pocket.

FIG. 5(A-D): LCN2 inhibitors reduce cell proliferation and cell viability in IBC cells. (A) For colony formation assays SUM149 cells were treated with LCN2 inhibitors at 10 μM, 1 μM, and 0.1 μM concentration. (B) The percentage of clonogenicity was calculated relative to DMSO. Results are shown as Mean±SEM of triplicate experiments (**P<0.01, ***P<0.001). (C) Representative plate of showing a colony formation assay of SUM149 cells treated with the LCN2 inhibitor. (D) Cell viability was assessed in SUM149 cells with Alamar Blue dye 72 hours after LCN2 inhibitor treatment. The percentage of cell viability was calculated relative to DMSO. Results are shown as Mean±SEM of triplicate experiments (****P<0.0001).

FIG. 6(A-B): LCN2 inhibitors ZINC00784494 and ZINC00640089 reduced p-Akt in a dose-dependent manner in SUM149 cells. SUM149 cells were incubated with each inhibitor as described in the “Materials and Methods” section. Changes in AKT and p-AKT protein levels were measured by western blot with specific antibodies againts these protein forms. (A) ZINC00784494, (B) ZINC00640089.

FIG. 7(A-C): LCN2-inhibitors ZINC00784494 and ZINC00640089 showed specificity toward LCN2-calyx. Cell proliferation in MCF7, MCF7-EV and. MCF7-LCN2 cells after treatment with (A) ZINC00784494 inhibitor and (B) ZINC00640089 inhibitor. (C) A representative clonogenic assay of MCF7, MCF7-EV, and MCF7-LCN2 treatment with ZINC00784494 and ZINC00640089. Results are shown as Mean±SEM of triplicate experiments (**P<0.01, ****P<0.0001).

FIG. 8(A-B): Effect of LCN2 silencing on cell viability of IBC cells. Cell viability was assessed in (A) MDA-IBC3 and (B) SUM149 with Alamar Blue dye 72 hours after negative control (NC-siRNA), LCN2-siRNA-1, and LCN2-siRNA-2 transfection. No significant difference was observed between LCN2-siRNAs and NC-siRNA. Results are shown as Mean±SEM of triplicate experiments.

FIG. 9: LCN2-siRNA-based silencing induces Caspase-3 activation in MDA-IBC3 cells. Caspase-3 fluorometric activity assay in MDA-IBC3 cells after LCN2-siRNA-2 and NC-siRNA transfection, and docetaxel treatment as positive control. Results are shown as Mean±SEM of triplicate experiments (**P<0.01, ***P<0.001).

FIG. 10(A-B): Assessment of cell cycle progression was performed by flow cytometry after LCN2-siRNA silencing in IBC cells. (A) Histogram showing a tendency in cell cycle arrest at G0/G1 to S phase transition 48 hours after LCN2-siRNA-2 transfection in SUM149 cells compared to NC-siRNA. (B) Quantification of flow cytometry data showing an increase in SUM-149-LCN2-siRNA-2 (41.8%) transfected cells at G0/G1 to S phase transition compared to NC-siRNA (34.2%).

FIG. 11(A-B): LCN2 inhibitors not showing reduction in cell proliferation in IBC cells. Colony formation assay was performed in SUM-149 cells after treatment with LCN2 inhibitors at 10 μM, 1 μM, and 0.1 μM. (A) 6 inhibitors; (B) 6 inhibitors. The percentage of clonogenicity was calculated relative to DMSO. Results are shown as Mean±SEM of triplicate experiments.

FIG. 12(A-P): Western blot images: (A) Western blot LCN2 in MCF-7 clones, (B) Western blot LCN2-FLAG-tagged in MCF-7 clones, (C) Western blot of LCN2 expression in breast cancer cell lines (IBC and non-IBC), (D) Western blot LCN2-siRNA-based silencing in MDA-IBC3 cell line, (E) Western blot LCN2-siRNA-based silencing in SUM149 cell line, (F) Western blot full length Caspase 3, (G) Western blot full length Caspase 9, (H) Western blot Cleaved Caspase 9, (I) Western blot PARP-1, (J) Western blot Cleaved PARP-1, (K) Western blot p21, (L) Western blot p27, (M) Western blot CDK4, (N) Western blot Cyclin E1, (O) Western blot Cyclin E2, (P) Western blot Cleaved Caspase 3.

FIG. 13: Crystal structure of Siderocalin (NGAL, Lipocalin 2) complexed with Fe-TrenCam-hopo2. PDB ID, 3HWG.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

Inflammatory Breast Cancer (IBC) is an aggressive form of invasive breast cancer, highly metastatic, representing 2-4% of all breast cancer cases in the United States. Despite its rare nature, IBC is responsible for 7-10% of all breast cancer deaths, with a 5-year survival rate of 40%. Thus, targeted and effective therapies against IBC are needed.

Recent efforts to identify specific therapeutic targets for IBC have led to the discovery of several non-specific inhibitors, some in the preclinical stage and others in ongoing clinical trials. For example, the combination of a histone deacetylase inhibitor (HDACi) with nanoparticle albumin-bound paclitaxel is part of the metastatic clinical trial to treat HER2-negative IBC.

The present application used a structure-based computational approach to identify potential LCN2 inhibitors in the ZINC database of the Asinex library. Based on molecular docking simulations, it was predicted that hit compounds binding to the LCN2-calyx pocket and interfering with key residues Trp79, Arg81, Tyr106, Lys125, and Lys134 would inhibit LCN2 activity and, thus, cell proliferation and viability. Moreover, because there is no evidence of LCN2 inhibitors in the literature, the small molecule inhibitors generated against LCN2 are disclosed for the first time.

The inhibitors proposed herein target the interacting region between LCN2 and its natural ligands, bacterial siderophores and catecholate. Because bacterial siderophores are iron carriers, the reduction in cell proliferation and cell viability of IBC cells upon drug treatment may be caused by an impairment in cellular activities due to a shortage in iron uptake. In fact, certain types of cancers can reprogram iron metabolism to allow cancer cells to survive. Although therapies to block iron dependencies have been extensively studied in cancer, there are no therapies to inhibit iron uptake by cancer cells in IBC. Therefore, the claimed invention herein represents a novel therapeutic approach for IBC.

Molecular docking simulations performed for ZINC00784494 and ZINC00640089 predicted that both compounds would bind to the LCN2-calyx pockets. Therefore, these compounds may inhibit LCN2's iron transporting ability mediated by its ligands. The siderophores, Trp79, Tyr106, and Lys106 are the likely key residues in the LCN2-calyx that interact with both inhibitors. The LCN2 ligand-contacting residues conserved across 18 vertebrate species, include Lys134, Trp79, and Tyr106, which form the three main pockets that define the calyx binding site.

Each of the three LCN2 residues interacting with the inhibitors are within this three-pocket assembly. Therefore, the LCN2-inhibitors identified may block the interaction between LCN2 and its natural ligands in vitro as well as in vivo. When inhibitor ZINC00784494 and inhibitor ZINC00640089 were tested against non-expressing-LCN2 breast cancer cells (MCF7) and LCN2-overexpressing clones (MCF7-LCN2), the inhibitors ZINC00784494 and ZINC00640089 significantly decreased MCF7-LCN2 cell proliferation, consistent with specificity of both inhibitors towards the LCN2-calyx. Moreover, the reduction of p-Akt levels after treatment of SUM149 cells with ZINC00784494 and ZINC00640089 inhibitors, further supports the specificity of these LCN2 inhibitors.

Because LCN2 plays a pivotal role in cancer, targeting this protein offers a novel opportunity to develop a specific IBC treatment drug. Inhibiting LCN2 using RNAi or small molecule inhibitors offers several advantages over the therapeutic regimens currently available. First, for IBC patients where HER2 or the epidermal growth family receptor (EGFR) are not expressed or mutated, targeting LCN2 is an option. Second, as LCN2 is overexpressed in IBC cells, small inhibitors or RNAi can be combined with chemotherapeutic agents commonly used for IBC treatment. Finally, using nanoliposomal formulations to deliver LCN2-siRNAs, or using LCN2 inhibitors targeting the LCN2-calyx, represent feasible approaches to develop new IBC treatments.

### Example 1

### LCN2 Protein Levels are Increased in Inflammatory Breast Cancer Cell (IBC) Lines

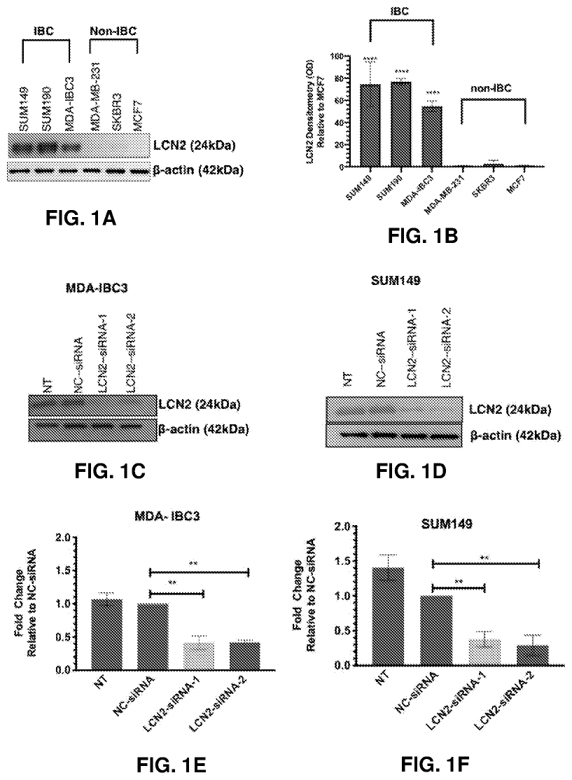

First, the LCN2 protein levels were measured in a panel of IBC and non-IBC cell lines. The description of the cell lines, including ER, PR, and HER-2 status, are shown in Table 1. Western blot analysis showed significantly higher LCN2 protein levels in IBC cells than non-IBC cells (FIG. 1A). Densitometric analysis of band intensities confirmed our findings (****, P<0.0001, FIG. 1B).

### Example 2

### LCN2-siRNA-Based Silencing Reduces Cell Colony Formation, Cell Invasion and Migration in IBC Cells

Two different small interference RNA (siRNA) molecules were used to silence human LCN2 (NC_000009.12): (1) LCN2-siRNA-1: target sequence: 5′GGAAUGCAAUUCUCAGAGA-3′ (SEQ ID NO: 1); (2) LCN2-siRNA-2: target sequence: 5′-CAUGCUAUGGUGUUCUUCA-3′ (SEQ ID NO: 2).

The biological effects of siRNA-mediated LCN2 silencing was determined in IBC cells, MDA-IBC3 (HER2+) and SUM149 (TNBC) IBC cells. Western blot analysis of MDA-IBC3 showed a significant decrease in LCN2 protein levels in cells transiently transfected with siRNAs compared to controls (FIG. 1C). Densitometric analysis of band intensities showed a decrease of LCN2 protein levels of 59% and 58% with LCN2-siRNA-1 and LCN2-siRNA-2, respectively (FIG. 1D, **P<0.01). Similar results were obtained when LCN2 was silenced in SUM149 cells (FIG. 1E). Densitometric analysis showed a decrease of LCN2 protein levels of 62% and 71% with LCN2-siRNA-1 and LCN2-siRNA-2, respectively (**P<0.01, FIG. 1F).

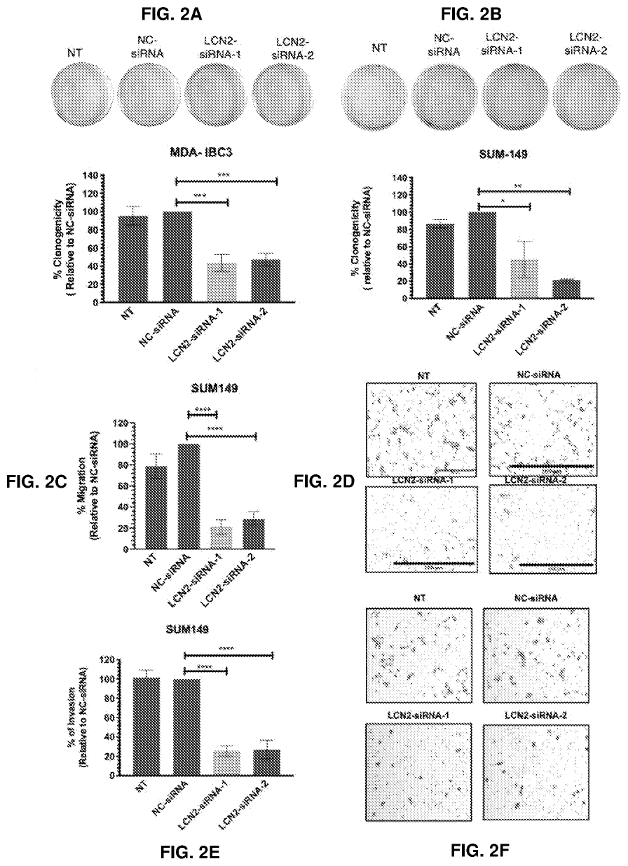

To assess the long-term effect of LCN2 silencing in IBC cells, colony formation assays were performed. Transient transfection of LCN2-siRNAs on MDA-IBC3 cells significantly reduced the number of colonies compared with the negative control siRNA (NC-siRNA) (LCN2-siRNA-1: 57%; LCN2-siRNA-2: 53% reductions on colony formation, **P<0.01, FIG. 2A). LCN2 silencing in SUM149 significantly decreased the number of colonies compared with the NC-siRNA (LCN2-siRNA-1: 45% and LCN2-siRNA-2: 79% reductions on colony formation, *P<0.05, **P<0.01, respectively; FIG. 2B).

Because overexpression of LCN2 has been associated with increased metastasis of cancer cells, the effect of LCN2 silencing in the migration and invasiveness potential of IBC cells was investigated. A significant reduction in the migration of SUM149 cells was observed following LCN2 silencing compared to NC-siRNA (LCN2-siRNA-1: 79% and LCN2-siRNA-2: 71% reductions; ****P<0.0001, FIG. 2C, 2D). In the transwell invasion assays, a significant reduction in the number of invaded cells was observed compared to NC-siRNA (LCN2-siRNA-1: 77% and LCN2-siRNA2: 71% reductions, ****P<0.0001, FIG. 2E, 2F). No visible changes in the short-term viability of MDA-IBC3 or SUM149 cells after LCN2 silencing (FIG. S1A, S1B) were found. This evidence suggests that LCN2-siRNA silencing has more pronounced effects on cell growth, proliferation and invasion than in cell viability of IBC cells.

### Example 3

### LCN2 Silencing Induces Apoptosis and Cell Cycle Arrest

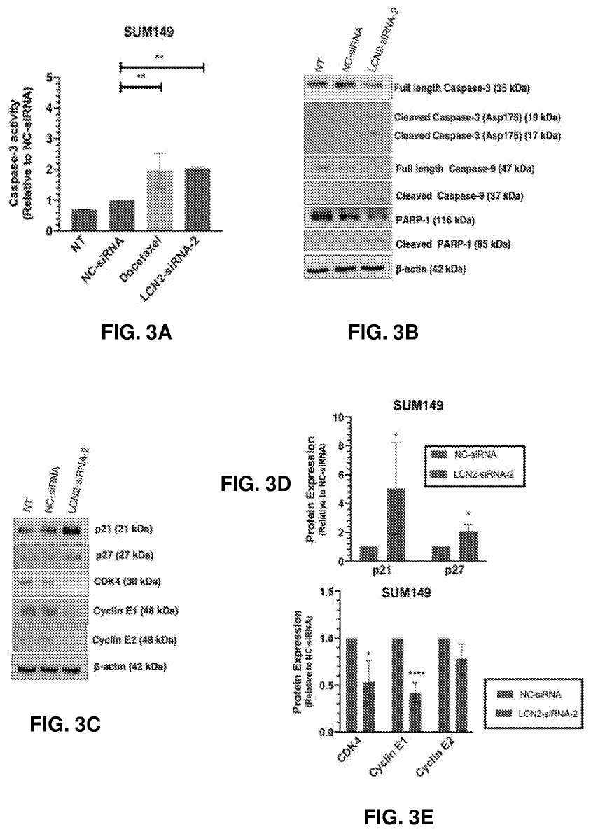

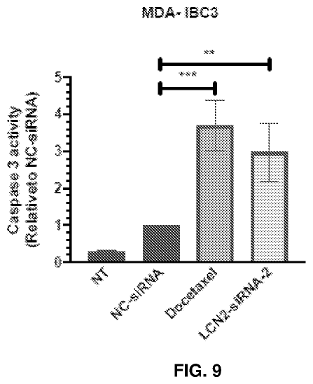

A question was whether the reduction in colony formation after LCN2 silencing was due to the activation of apoptosis, cell cycle arrest, or both. The activation of caspase-3 was measured as the indicator of apoptosis. Compared with NC-siRNA, silencing of LCN2 with siRNAs in SUM149 cells resulted in a 2-fold increase in Caspase-3 activity (**P<0.01, FIG. 3A). Similar results were observed after LCN2 silencing in MDA-IBC3 cells, where around a 3-fold increase in caspase-3 activity was observed (FIG. 9). Docetaxel, a common drug used for IBC treatment, used here as a positive control, also resulted in a two-fold increase of caspase-3 activity.

Activation of apoptosis was confirmed measuring changes in apoptotic-related proteins by Western blot analysis. Transient transfection of LCN2-siRNA-2 achieved a reduction of full-length Caspase-3 and full-length Caspase-9, and a significant increase in the active form of Caspase-9 (cleaved Caspase-9) and Caspase-3 (cleaved Caspase-3). Moreover, a reduction in the poly-ADP ribose polymerase-1 (PARP-1) full-length band intensity, together with an increase in the cleaved PARP-1 band compared with NC-siRNA, was observed (FIG. 3B).

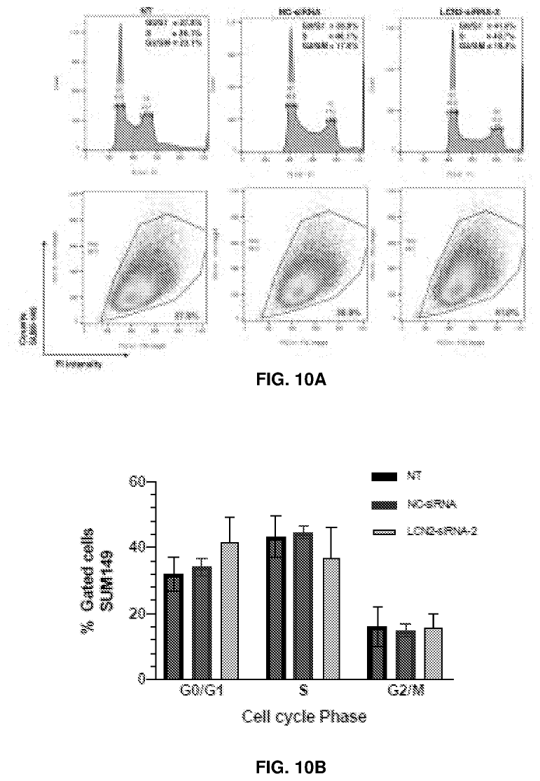

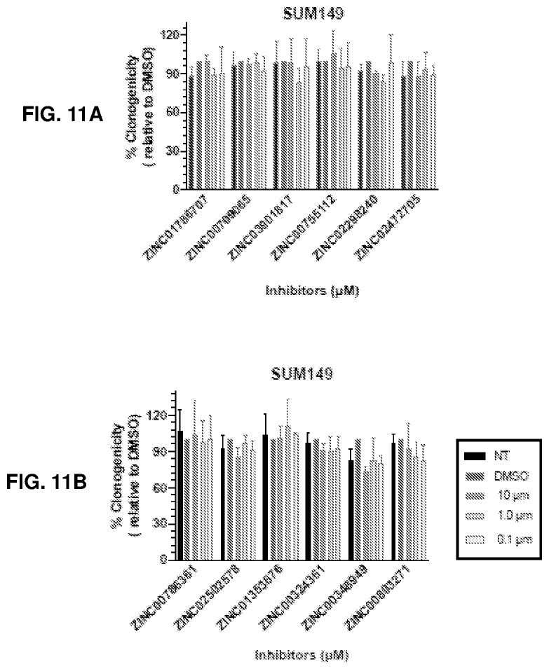

Cell cycle progression was measured after LCN2 silencing in SUM149 cells by flow cytometry. Cell cycle arrest in the G0/G1 to S phase was observed in SUM149 cells, 72 hours post-transfection (**P<0.01, ***P<0.001, FIG. 3C, 3D). These results were confirmed by studying the changes of key proteins involved in the G0/G1 to S phase transition by western blot analysis. Notably, an increase in the protein levels of tumor suppressors p21 and p27 was observed (*P<0.05, FIG. 3E, 3F). Moreover, a reduction of the checkpoint proteins of S phase—cyclin E1, cyclin E2, and CDK4—was observed (*P<0.05, ****P<0.0001, FIG. 3E, 3G). Compared with NC-siRNA, transient transfection of LCN2-siRNA-2 in SUM149 cells showed a tendency in cell cycle arrest in G0/G1 to S phase, 48 hours post-transfection (FIG. 10A-B).

### Example 4

Identification of LCN2 Small Molecule Inhibitors by in-Silico Analysis

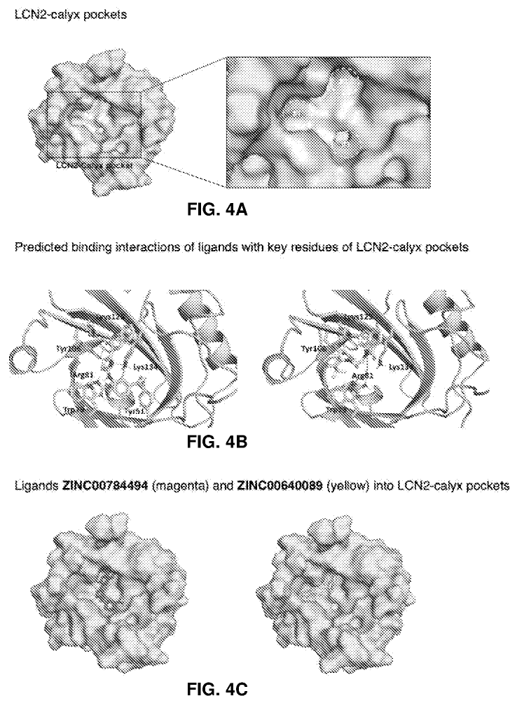

To identify lead compounds that potentially target LCN2, the structural properties of the crystal structure of the LCN2-calyx pocket and ligand-bound structures were analyzed. The LCN2-calyx includes three pockets (Pockets #1, #2, and #3 of FIG. 4A) that accommodate critical functional groups for siderophores, which creates specificity for ligand recognition. The key siderophore-contacting residues are Trp79, Arg81, Tyr106, Lys125, and Lys134. Moreover, the side chains of residues Trp79 and Arg81 increase flexibility at the LCN2-calyx allowing the accommodation of different ligands in the protein pocket.

Structure-based virtual screening and molecular docking studies were carried out between the LCN2 protein and a set of 25,000 ligands from the Asinex library using the PyRx virtual screening tool (Dallakyan S., et al. Methods Mol. Biol. 2015:1263; 243-250). After docking these compounds into LCN2, the results display various modes of ligand-receptor interactions generated with a docking score. With a binding energy cut-off of −9.6 kcal/mol, a total of 265 hits (1.1% of total ligands) were identified, with the least binding energy ranging from −11.5 to −9.6 kcal/mol. The ligands with a binding energy of −9.6 kcal/mol or less were visualized using the PyMol molecular graphics system. (Schiffrin B. et al., Protein Sci. 2020 29 (8) 1851-1857). Next, the selected 265 ligands were submitted to the Swiss-ADME server, and the list of the best candidates was refined according to the drug-likeness score using the Lipinski rule of five, physicochemical properties, lipophilicity, water-solubility, pharmacokinetics, and the pan-assay interference (PAINS) filter for the identification of potentially problematic fragments (Table 2). The structure-based screening resulted in the selection of 138 molecules with a binding energy range between −11.5 to −10.0 kcal/mol. The 138 ligands were re-ranked based on structural characteristics, predicted binding geometries (docking poses) using PyMOL, and on the main interactions between key residues at the binding site of the LCN2-calyx pocket and the selected ligands. The presence of polar interactions of ligands with Trp79, Arg81, Tyr106, Lys125, and Lys134 was used as a selection criterion, in addition to other polar interactions and stereochemical complementarity. Finally, a total of 25 ligands were selected with a binding affinity ranging between −11.5 to −10.3 kcal/mol, from which 16 ligands were commercially available and used for further in vitro analysis (Table 3). Results of the docked complexes indicated that the binding sites of these ligands interact between side chains of residues of the LCN2-calyx pocket (FIG. 4B, 4C).

According to FIG. 4B, the predicted binding of ligand ZINC00784494 with LCN2-calyx (−10.4 kcal/mol) was through hydrogen-bonding of the thiazole ring with the side chain —NH of Lys134, and the carbonyl group of the chromenone moiety with the phenol group of Tyr106 residue, and hydrophobic x-interactions of the phenyl ring with Trp79 indole group (FIG. 4B).

Similarly, compound ZINC00640089 binds to LCN2-calyx (−10.6 kcal/mol) by positioning the 2-oxo-benzoindole ring near Lys 134 for possible hydrogen bonding between the carbonyl group and the-NH group. The carbonyl group of the acetamide moiety of ligand ZINC00640089 is also in proximity for favorable hydrogen bonding with the phenolic group of Tyr106 (FIG. 4B). As seen from the docking representations as surface models (FIG. 4C), both compounds occupy two pockets into the LCN2-calyx binding site, predicting the potential to block the interaction between LCN2 with its natural ligands.

### Example 5

LCN2-inhibitors Reduce Colony Formation and Cell Viability in SUM149 Cells

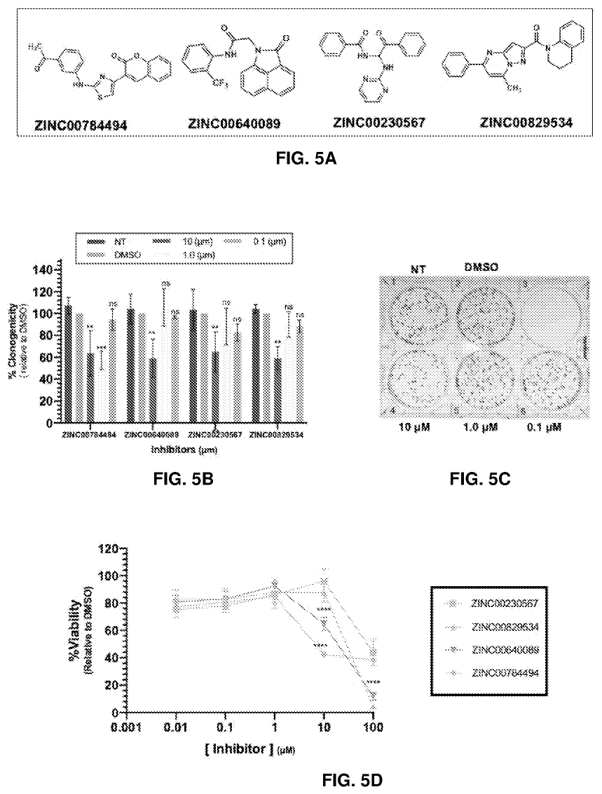

Clonogenic assays were used to investigate the effect of the selected compounds on the self-renewing capacity of SUM149 cells. Sixteen compounds were selected through structure-based screening (Table 3). SUM149 cells were seeded and 24 hours later they were treated with each inhibitor at different concentrations (10 μM, 1 μM and 0.1 μM). Four out of the 16 compounds significantly decreased the number of colonies formed (FIG. 5A). The four selected small molecules were

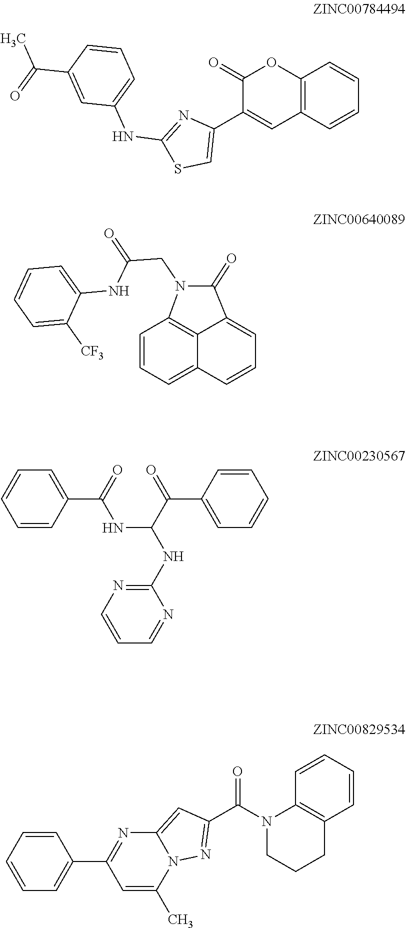 [SMILES: CC(=O)c1cccc(Nc2nc(-c3cc4ccccc4oc3=O)cs2)c1 ; O=C(CN1C(=O)c2cccc3cccc1c23)Nc1ccccc1C(F)(F)F ; O=C(NC(Nc1ncccn1)C(=O)c1ccccc1)c1ccccc1 ; Cc1cc(-c2ccccc2)nc2cc(C(=O)N3CCCc4ccccc43)nn12]

Particularly, the compound ZINC00784494 showed a significant decrease in the number of colonies formed at 10 μM (37% reduction), and 1 μM (43% reduction) compared to Dimethyl sulfoxide (DMSO) (0.20%, final concentration) (**P<0.01, ***P<0.001, FIG. 5B). Compounds ZINC00784494, ZINC00640089, ZINC00230567, and ZINC00829534 significantly reduced the number of colonies formed at 10 μM (42%, 62%, and 41% reduction, respectively) compared to DMSO (**P<0.01, ***P<0.001, FIG. 5B). Any of the four compounds significantly reduced the number of colonies at concentrations of 1 μM or 0.1 μM (FIG. 5B). FIG. 5C is a representative plate showing the changes observed in colony formation with the compounds ZINC00784494, ZINC00640089, ZINC00230567, and ZINC00829534. The additional 12 compounds tested did not show changes in cell proliferation at any of the tested concentrations FIG. 11(A-B).

Cell viability of SUM149 cells were further tested with the four compounds that significantly reduced the number of colonies (FIG. 5D). As compared with DMSO, the four compounds significantly reduced cell viability at 100 μM (60% reduction, ****P<0.0001,FIG. 5D). Inhibitors ZINC00784494 and ZINC00640089 significantly reduced cell viability at 10 μM. (57%, 35% reduction, respectively, ****P<0.0001, FIG. 5D). Any of the four compounds reduced cell viability at concentrations of 1 μM or lower. (FIG. 5D).

### Example 6

The LCN2 Inhibitors ZINC00784494 and ZINC00640089 Reduced the p-Akt Levels in SUM149 Cells

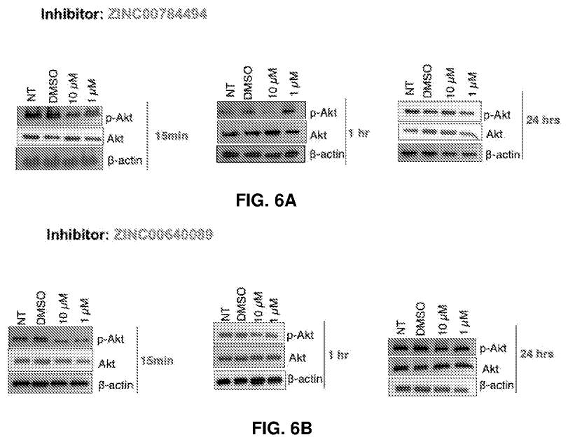

Evidence indicates that LCN2 activates the EGFR/AKT, a critical pathway regulating the growth, survival, proliferation, and differentiation of mammalian cells. Therefore, the effect of LCN2 inhibitors in the phosphorylation levels of AKT. SUM149 cells were treated with the LCN2 inhibitors ZINC00784494 and ZINC00640089 at 10 μM and 1 μM. NT cells and cells treated with DMSO (0.2% final concentration) were used as controls. As compared to DMSO, 10 μM and 1 μM of the LCN2 inhibitor ZINC00784494 reduced the p-Akt protein levels 15 minutes and 1 hour after drug treatment (FIG. 6A). Changes in the p-Akt protein levels were not observed 24 hours after drug treatment (FIG. 6A). Similarly, a reduction in p-AKT protein levels was observed at 10 μM and 1 μM concentrations with the LCN2 inhibitor ZINC00640089. The total Akt protein levels were unaltered in all of the dose and time-points tested (FIG. 6A, 6B).

### Example 7

### LCN2-Inhibitors ZINC00784494 and ZINC00640089 Showed Specificity Toward LCN2

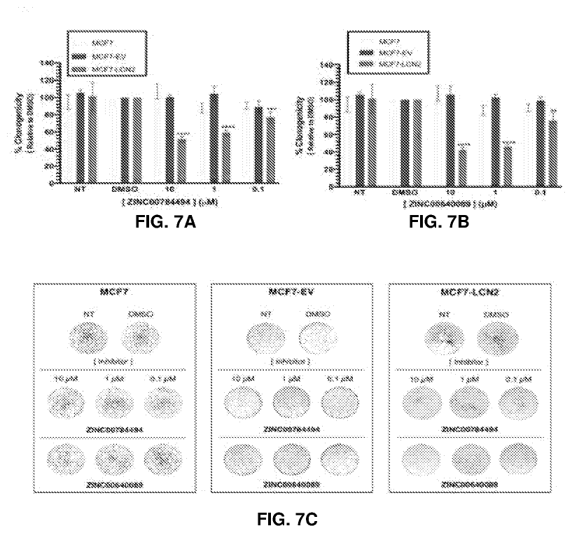

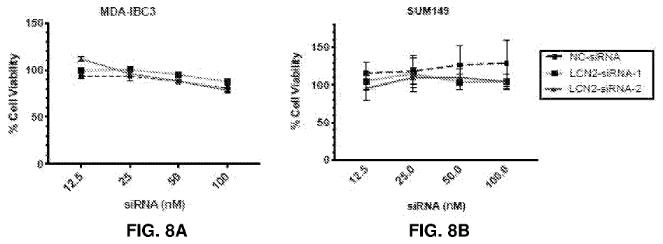

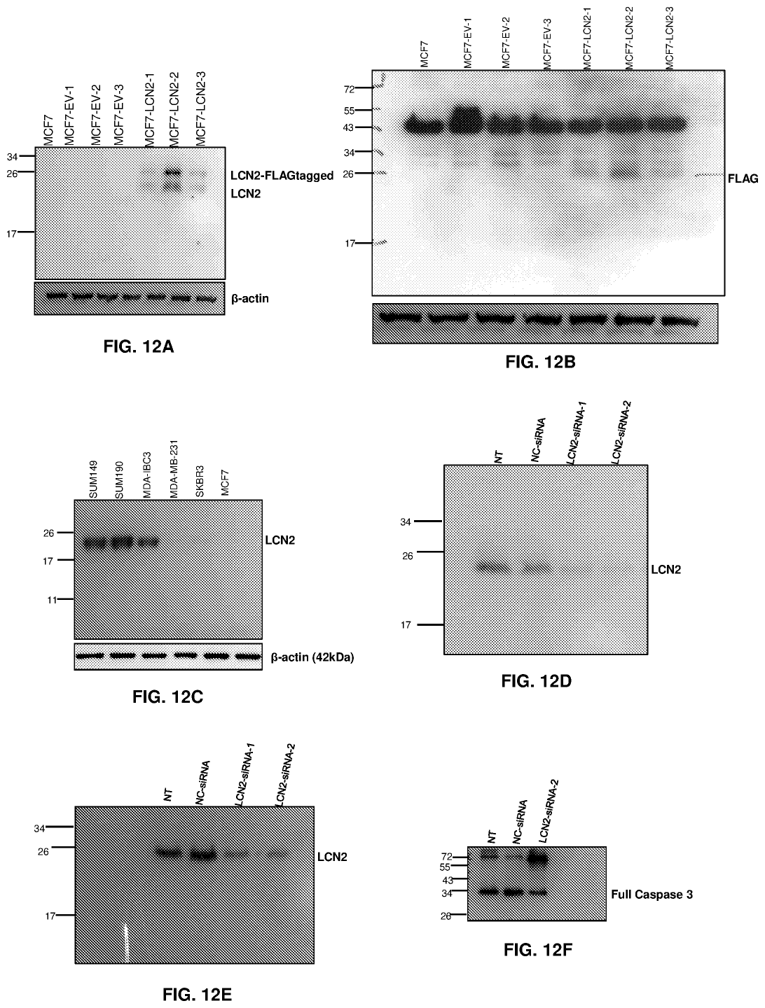

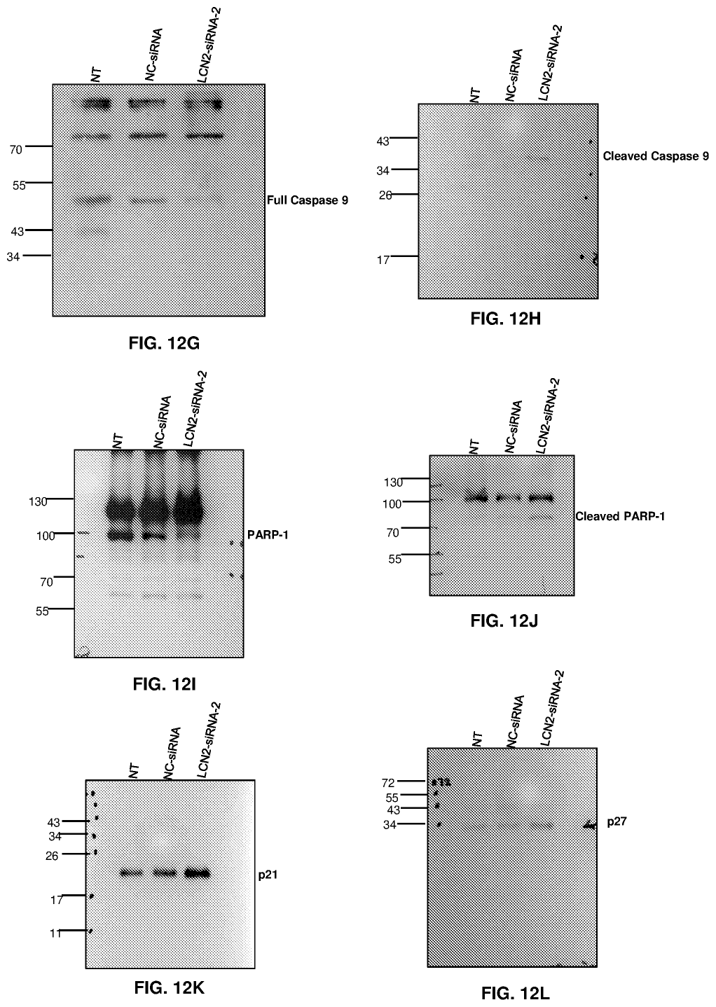

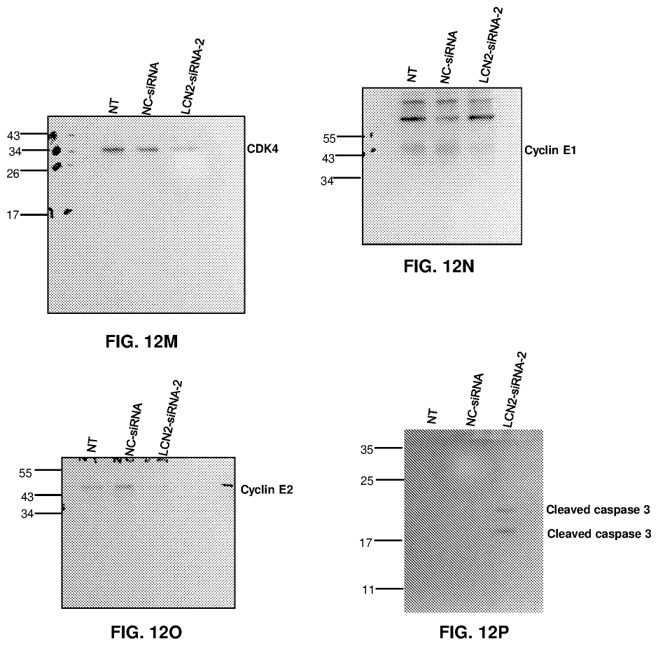

The selectivity of the compounds towards LCN2, MCF7, ectopically expressing LCN2 were exposed to ZINC00784494 and ZINC00640089 inhibitors. FIG. 12(A-P) shows the LCN2 expression in MCF7, MCF7-EV, and MCF7-LCN2 cells. For comparison purposes, all cells were treated with DMSO at 0.2%. A significant reduction in colony formation of MCF7-LCN2 cells treated with ZINC00784494 at 0.1 μM, 1 μM, and 10 μM concentration were observed when compared with untreated cells (23%, 41%, and 49%, decrease respectively, ***P<0.001, ****P<0.0001, FIG. 7A). The same concentrations of the inhibitor did not cause any effect on MCF7-EV cells (FIG. 7A). Similar effects were observed with the inhibitor ZINC00640089 at 0.1 μM, 1 μM, and 10 μM concentration were observed when compared with untreated cells (24%, 54%, and 57%, decreases respectively, **P<0.01, ****P<0.0001, FIG. 7B). FIG. 7C shows the changes observed in the clonogenic assays after treatment of MCF7, MCF7-EV, and MCF7-LCN2 cells with the inhibitors ZINC00784494 and ZINC00640089. Together, these results suggest that ZINC00784494 and ZINC00640089 inhibit LCN2.

### Discussion

LCN2 is significantly overexpressed in IBC cells compared to non-IBC cells. LCN2-siRNA silencing reduced colony formation, migration, and invasiveness ability of IBC cells. Moreover, targeting LCN2-calyx with small molecule inhibitors decreased colony formation and cell viability of IBC cells. Thus, results support LCN2 as a potential therapeutic target for IBC.

The present disclosure reveals that LCN2 silencing achieved a significant decrease in proliferation, migration, and invasion of IBC cells. The observed reduction in the number of colonies upon LCN2 silencing supports that LCN2 promotes the self-renewal capacity of IBC tumor cells. Similarly, the reduction of the invasion ability of SUM149 following LCN2 silencing supports a role of LCN2 in the epithelial to mesenchymal transition (EMT) process, a characteristic of the highly metastatic IBC cells. Reports indicate that a hybrid epithelial/mesenchymal (E/M) phenotype occurs in IBC cells. This hybrid E/M phenotype may promote IBC cells clustering together, forming circulating tumor cells (CTCs). CTCs possess a highly metastatic potential and contribute to metastasis.

Cell cycle arrest was found in the G0/G1 to S phase transition following LCN2 silencing in SUM149 cells. The arrest in cell cycle progression at the G0/G1 to S phase transition was confirmed by increases in the levels of cell cycle inhibitory proteins, p21 and p27, and the decrease in cyclin E1, cyclin E2, and CDK4. Moreover, the changes observed in the apoptotic markers, caspase-3, caspase-9 and PARP-1 indicate that LCN2 silencing activates both cell cycle progression arrest and apoptosis.

### Materials and Methods

A. Cell culture

The human IBC cell lines MDA-IBC3 (estrogen receptor and progesterone receptor negative; HER2 positive), SUM149 (estrogen receptor and progesterone receptor negative; HER2 negative) were kindly donated by Dr. Bisrat Debeb from the Department of Breast Medical Oncology at MD Anderson Cancer Center, Houston, TX. Cells were cultured in Hams F-12 medium (Thermo Fisher Scientific) supplemented with heat-inactive 10% fetal bovine serum (FBS) (Thermo Scientific, Logan, UT, USA), 0.1% penicillin/streptomycin (Thermo Scientific), 5 μg/mL insulin from bovine pancreas (Sigma), and 1 μg/mL hydrocortisone (Sigma). Breast cancer cell (BCC) lines MDA-MB-231 (ATCC HTB-26), and SKBR3 (ATCC HTB-30) were purchased from American Type of Culture Collection (ATCC) and cultured in Dulbecco's modified Eagle's medium (DMEM) supplemented with heat-inactive 10% fetal bovine serum (FBS) (Thermo Scientific), and 0.1% penicillin/streptomycin (Thermo Scientific). BCC line MCF7 (ATCC HTB-22D) was cultured in Dulbecco's Modified Eagle Medium (DMEM) supplemented with heat-inactive 10% FBS (Thermo Scientific) and 0.1% penicillin/streptomycin (Thermo Scientific). Cell lines were grown at 37° C. and 5% CO₂. Experiments were performed at 75% to 85% confluence. Mycoplasma-free cells were always used.

### B. Western Blot Analysis

Cell lysates were collected on ice using lysis buffer (1% Triton X, 150 mM NaCl, 25 mM Tris HCl,0.4 mM NaVO₄, 0.4 mM NaF and protease inhibitor cocktail from Sigma, St. Louis, MO, USA) and vortexed periodically for 30 min. Lysates were centrifuged for 15 min at 4° C., and supernatants were collected. Total protein concentrations were determined using Bio-Rad DC Protein Assay reagents (Bio-Rad) following the manufacturer's protocol. Equal amounts of protein for each sample (40 μg to 50 μg per lane) were separated by SDS-PAGE, blotted onto nitrocellulose membranes, blocked with 5% non-fat milk, and probed with the appropriate dilution of the corresponding primary antibody. Once incubated with the primary antibody, membranes were rinsed and incubated with the corresponding HRP-conjugated secondary antibody. Bound antibodies were detected using an enhanced chemiluminescence substrate followed by autoradiography using a FluorChem™ 8900 (Alpha Innotech Corporation, San Leandro, CA, USA). Primary antibodies: anti-LCN2 (AF1757) (24 kDa), (R&D System, MN, USA); Caspase 9 (9502) (47 kDa), Caspase 3 (9665) (35 kDa), Cleaved Caspase 9 (20750) (37 kDa), Cleaved Caspase 3 (9664) (17/19 kDa), PARP-1 (46D11) (89,116 kDa), CDK4 (D9G3D) (30 kDa), CDK6 (DCS83) (36 kDa), Cyclin E1 (HE12) (48 kDa), Cyclin E2 (4132) (48 kDa), p21/Waf1/Cip1 (12D1) (21 kDa), p27/Kip1 (D69C12) (27 kDa), Akt (4685) (60 kDa), p-Akt (Ser473) (4060) (60 kDa) (Cell Signaling, Danvers, MA, USA); anti-β-actin (42 kDa) (Sigma). Secondary antibodies: anti-goat IgG horseradish peroxidase (HRP) (HAF 109) (R&D Systems), anti-mouse and anti-rabbit IgG (HRP) (Cell Signaling).

### C. Transient and Stable Transfections

Two different small interference RNA (siRNA) molecules were used to silence human LCN2 (NC_000009.12). (1) LCN2-siRNA-1: target sequence: 5′-GGAAUGCAAUUCUCAGAGA-3′ (SEQ ID NO: 1); and LCN2-siRNA-2: target sequence: 5′-CAUGCUAUGGUGUUCUUCA-3′ (SEQ ID NO: 2), and a scrambled universal negative control siRNA (NC-siRNA) (SIC001) (Sigma) were transiently transfected at a final concentration of 100 nmol/L. A non-treated (NT) cells (containing transfection reagent, only) were also used. MDA-IBC3 cells (5.5×10⁴ cells/mL) or SUM149 (5.0×10⁴ cells/mL) were seeded in Petri dishes and twenty-four hours later, siRNAs were mixed with Lipofectamine 2000 RNAiMax transfection reagent (Life Technologies) at a 1:3 (v/v) (MDA-IBC3) or 1:1 (v/v) (SUM149) ratio (siRNA: transfection reagent) in serum and antibiotic-free Opti-MEM medium (Life Technologies). The transfection mix was incubated for 20 min at room temperature (RT) and then added to the cells. Cells were incubated at 37° C. and collected 24 hours (MDA-IBC3 cells) or 48 hours (SUM149) after transfection. Transfected cells were used to verify the LCN2 silencing or for in vitro experiments.

Ectopic LCN2 expression was performed in breast cancer MCF7 cells. Human LCN2 open reading frame (LCN2ORF) (RC207685, OriGene, Rockville, MD) or empty vector pCMV6-Entry (MCF7-EV) (PS100001, OriGene, Rockville, MD was stable transfected into MCF7 cells. MCF7 cells (4.0×10⁴ cells/mL) were seeded in 6-well plates and incubated at 37° C. Twenty-four hours later, 5 μg of LCN2-ORF were mixed with Lipofectamine 2000 RNAiMax transfection reagent (Life Technologies) at a 1:1 (v/v) (plasmid: transfection reagent) in serum and antibiotic-free Opti-MEM medium (Life Technologies) and incubated at 37° C. Six hours later medium was replaced with MCF7 culture media and incubated at 37° C. Forty-eight hours later the antibiotic neomycin was added at a final concentration of 1.8 mg/mL for the selection of transduced MCF7 cells. After 2-3 weeks, independent colonies were picked and cultured separately as independent clones.

### D. Colony Formation Assays

Cell growth was assessed by colony formation assays: MDA-IBC3 cells (5.5×10⁴ cells/mL) or SUM149 (5.0×10⁴ cells/mL) were seeded into 6-well plates. Twenty-four hours later, siRNAs were added to the cells. Twenty-four hours (MDA-IBC3 cells) or forty-eight hours (SUM149) after transfection, 1500 cells for MDA-IBC3 and 1000 cells for SUM149 were seeded into 10-cm Petri dishes containing Hams F-12 (10% FBS, 0.1% antibiotic/antimycotic solution, 0.001% insulin from bovine pancreas, and 0.005% hydrocortisone), and incubated at 37° C. Twelve days (SUM149 cells) or 19 days (MDA-IBC3 cells) later, colony-forming cells were stained with 0.5% crystal violet solution. Colonies (with at least 50 cells) were counted under a light microscope (Olympus CKX41) in five random fields with a total magnification of 10×.

### E. Cell Viability Assays

MDA-IBC3 cells (5.5×10⁴ cells/mL) or SUM149 (5.0×10⁴ cells/mL) were seeded in 96-well plates. Twenty-four hours later, cells were transiently transfected with serial dilutions of LCN2-siRNA and NC-siRNA (12.5 nM, 25 nM, 50 nM, and 100 nM final concentrations) with Lipofectamine 2000 RNAiMax. Seventy-two hours after transfection, the medium was removed, and cell viability was measured using Alamar blue dye (Invitrogen, CA, USA) as previously described by [5]. Optical density (OD) values were obtained spectrophotometrically in a plate reader (Bio-Rad) after 3 hours of dye incubation. In all cases, percentages of cell viability were obtained after blank OD subtraction, taking the untreated cells values as a normalization control.

### F. Invasion and Migration Assays

Cell invasion and migration were assessed using the transwell assay. SUM149 (5.0×10⁴ cells/mL) were seeded into a 6-well plate and transfected with siRNAs as described for the colony formation assays. Forty-eight hours after transfection, cells were collected and resuspended in serum-free Hams F-12 at 5.0×10⁴ cells/mL. Fifty-five μL of Matrigel (Corning, Lowell, MA) was added to the upper part of the transwell chamber of a 24-well plate (BD Biosciences, San Diego, CA; 8-μm pore size) and incubated at 37° C. for 1 hour (for invasion assay). Two hundred μL of cells were placed on top of Matrigel of each upper chamber. The lower chamber of the transwell was filled with 200 μL Hams F-12 media (10% FBS) and the plate was incubated at 37° C. for 24 hours. The numbers of invaded or migrated cells were calculated as previously described.

### G. In Vitro Testing of Small Molecules Inhibitors

For cell viability, SUM149 (5.0×10⁴ cells/mL) were seeded in 96-well plates. Twenty-four hours later, cells were treated with serial dilutions (0.01 μM, 0.1 μM, 1.0 μM, 10 μM, and 100 μM, final concentrations) of the LCN2 inhibitors. Seventy-two hours after treatment, cell viability was performed as above described, taking the DMSO (1%) treated OD values as the normalization control. For colony formation assays, SUM149 cells (5.0×10⁴ cells/mL), MCF7 (4.5×10⁴ cells/mL), MCF7-LCN2 (4.5×10⁴ cells/mL), or MCF7-EV (4.5×104 cells/mL) were seeded into 24-well plates and incubated at 37° C. Twenty-four hours later, LCN2 inhibitors bought to Asinex corporation (North chestnut, NC) were dissolved in DMSO (0.2%) and added to the cells at 10 μM, 1 μM, and 0.1 μM (final concentrations). NT cells and DMSO (0.2%) treated cells were used as controls. SUM149 (500 cells) and MCF7 (3,000 cells) cells were seeded in 6-well plates per treatment for colony formation assays.

### H. Akt/p-Akt Measurements

SUM149 cells (5.0×10⁴ cells/mL) were seeded in 10-cm Petri plates and incubated at 37° C. Twenty-four hours later, cells were treated with the LCN2 inhibitors ZINC00784494 and ZINC00640089 at 10 μM and 1 μM concentrations. NT and DMSO (0.2% final concentration) treated cells were used as controls. Cell pellets of each condition were collected at 15 minutes, 1 hour, and 24 hours after drug treatment. The p-AKT Akt and Akt protein levels were assessed by Western blots as above described.

### I. Caspase-3 Fluorometric Assay

Caspase-3 activity was assessed using a caspase-3/CPP32 fluorometric assay kit (Bio Vision, CA, USA) as described in the manufacturer's protocol, with some modifications. Briefly, SUM149 (5.0×104 cells/mL) and MDA-IBC3 (5.5×104 cells/mL) were seeded in 10 cm Petri dishes and transiently transfected with siRNAs as above described. Docetaxel (0.5 nM final concentration) was used as a positive control. Seventy-two hours post-transfection cells were collected, protein extracts were obtained and incubated with the Asp-Glu-Val-Asp (DEVD) peptide substrate—which is conjugated to the 7-amino-4-trifluoromethyl coumarin (AFC)—at 37° C. for 60 min. Releasing of AFC was measured with a fluorometric plate reader (Varioskan LUX, ThermoFisher, USA) at an excitation wavelength of 400 nm and an emission wavelength of 505 nm. The fold-change increase in caspase-3 activity was determined by comparing the release of AFC from the siRNA-transfected cells with the AFC release by the untreated cells.

### J. Flow Cytometry Analysis

To assess cell cycle progression, SUM149 cells were transfected with siRNAs as described above. Forty-eight and seventy-two hours later, attached cells were collected, washed in ice-cold PBS, fixed with 70% cold ethanol, and stored at 4° C. Twenty-four hours later, cells were washed with ice-cold PBS, resuspended in propidium iodide (PI)/RNase Staining Buffer (BD Biosciences), incubated in the dark for 15 min at room temperature, and then analyzed by flow cytometry in FACS Calibur (BD Biosciences). FLOWJO Software (BD Biosciences) was used to determine the percentage of cells in each phase of the cell cycle.

### K. Molecular Docking

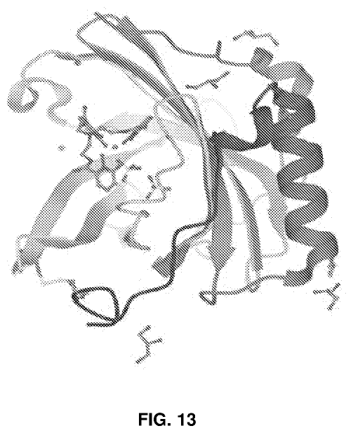

PyRx virtual screening tool version 0.8 which uses AutoDock Vina and AutoDock 4 as a docking software was used with the Lamarckian genetic algorithm as scoring function for higher docking accuracy, AutoDockTools to generate input files, and Phyton as a programming/scripting language. The target protein Lipocalin 2 (crystal structure of the macromolecule, X-ray diffraction 2.19 Å, FIG. 13) was retrieved from the Protein Data Bank web site as a PDB file format and prepared using AutoDock 4 by eliminating water molecules and the bound ligands from the calyx binding site and saved as PDBQT file format. Asinex screening library of 25,000 ligands was used to retrieve 2D structures in SDF file format, and further using Open Babel software, these compounds were converted to PDB file format. The grid box coordinates (Vina Search Space) were located at the center of Lys134 with dimensions (Å) of X: 30.0925, Y: 760246, Z: 60.8653 to dock all the ligands where 8 maximum exhaustiveness was calculated for each ligand. Lys 134 is a key residue located at pocket #2 that has been identified to form hydrogen bonding interactions with the siderophores of LCN2. All other parameters of software were kept as default, and all bonds contained in ligands were allowed to rotate freely and considering macromolecule as rigid. The results from the PyRx tool and virtual screening are obtained as CSV or SDF file format for further analysis and data organization. The final visualization of the docked structure was performed using PyMOL v2.4 (www.pymol.org) and was used for protein alignment and as molecular viewer to generate high-quality molecular structures.

### L. Physicochemical and Pharmacokinetic Properties

Physicochemical and pharmacokinetic properties including absorption, distribution, metabolism (ADME), lipophilicity, water solubility, drug likeness, and the PAINS model, were predicted using SwissADME server. The physicochemical properties values are computed using OpenBabel v2.3.0. The lipophilicity is calculated using five predicted models including XLOGP3, WLOGP, MLOGP, SILICOS-IT, and iLOGP. The water solubility is calculated by the server using the ESOL model and a modified version of the general solubility equation (GSE) model. The pharmacokinetic properties adapt the support vector machine (SVM) algorithm to estimate substrate for the P-gp or inhibitor for the most important CYP isoenzymes. The Lipinski rule of five filter used for drug-likeness prediction is implemented from (Lipinski, et al., Adv Drug Deliv Rev. 2001 Mar. 1; 46(1-3): 3-26). The rule of ‘5’ state that poor absorption and low membrane permeation occur when hydrogen-bond donors are ≥5, MW≥500, LogP>5, and hydrogen-bond acceptors≥10. The PAINS model implements a rule-based method for lead-likeness, which was adapted from references Robertson et al. CA Cancer J. Clin. 2010:60 (6):351-375 and Baell, JB, et al., J. Med. Chem. 2010, 53, 2719-2740. The methods for PAINS calculation were implemented using the SMARTS recognition capability of OpenBabel API. The SMARTS definitions for PAINS were retrieved from the Filter-it distribution (version 1.0.2, 2013, http://silicos-it.be.s3-website-eu-west-1.amazonaws.com/software/filter-it/1.0.2/filter-it.html).

### M. Statistical Analysis

All experiments were performed at least in triplicate. Graphs were constructed with the GRAPH PAD Prism 8 software (GraphPad Software, Inc., La Jolla, CA). Data were analyzed using Student's t-test for comparing two groups and ANOVA tests for multiple group comparisons, with p<0.05 considered statistically significant (* p<0.05, ** p<0.01, p<0.001).

TABLE 1
Receptor expression in IBC and non-IBC cell lines.
Inflammatory Breast Cancer Cell Lines: IBC-3
(PR−, ER−, HER2+), SUM-149 (PR−, ER−,
HER2−). Non-Inflammatory Breast Cancer Cell Lines:
metastatic (MDA-MB-435 (ER−, PR,
HER2+), MDA-MB-231 (ER−, PR−, HER2−), and
non-metastatic (MCF7 (ER+, PR+, HER2−).

| | Markers | | | |
|---|---|---|---|---|
| Breast Cancer Lines | ER | PR | HER2 | |
| IBC | | | | |
| SUM-149 | − | − | − | Metastatic |
| MDA-IBC3 | − | − | − | Metastatic |
| NON-IBC | | | | |
| SKBR-3 | − | − | + | Metastatic |
| MDA-MB-231 | − | − | − | Metastatic |
| MDA-MB-435 | − | − | + | Metastatic |
| MCF7 | + | + | − | Non-Metastatic |

TABLE 2
Predicted physicochemical, lipophilicity, water solubility, druglikeness, and
PAINS properties of LCN2 inhibitors and compound derivatives.

| | Physicochemical Properties | | | | | | | | | | |
|---|---|---|---|---|---|---|---|---|---|---|---|
| | | | | H- | | | | | Druglikeness | | |
| | | | Rotatable | bond | H-bond | Lipophilicity | Water solubility | | | Bioavailability | |
| Molecule ID | MW^{a} | TPSA^{b} | bonds | acceptors | donors | (LogP) | LogS | Solubility | Lipinski^{c} | score | PAINS^{d} |
| ZINC00829534 | 368.43 | 50.5 | 3 | 3 | 0 | 3.81 | −5.08 | Moderately | 0 | 0.55 | 0 |
| | | | | | | | | soluble | | | |
| ZINC02472705 | 312.32 | 58.89 | 1 | 3 | 1 | 3.97 | −5.19 | Moderately | 0 | 0.55 | 0 |
| | | | | | | | | soluble | | | |
| ZINC02298240 | 313.31 | 56.24 | 1 | 4 | 0 | 4.15 | −5.37 | Moderately | 0 | 0.55 | 0 |
| | | | | | | | | soluble | | | |
| ZINC00348949 | 312.32 | 47.51 | 1 | 3 | 0 | 3.72 | −5.36 | Moderately | 0 | 0.55 | 0 |
| | | | | | | | | soluble | | | |
| ZINC03901817 | 353.33 | 71.52 | 1 | 4 | 0 | 3.03 | −4.57 | Moderately | 0 | 0.55 | 1 |
| | | | | | | | | soluble | | | |
| ZINC00640089 | 370.32 | 51.1 | 5 | 5 | 1 | 4.08 | −4.86 | Moderately | 0 | 0.55 | 0 |
| | | | | | | | | soluble | | | |
| ZINCO2502578 | 338.36 | 51.96 | 3 | 3 | 0 | 3.83 | −4.87 | Moderately | 0 | 0.55 | 0 |
| | | | | | | | | soluble | | | |
| ZINC00230567 | 332.36 | 83.98 | 7 | 4 | 2 | 2.35 | −4.23 | Moderately | 0 | 0.55 | 0 |
| | | | | | | | | soluble | | | |
| ZINC00324361 | 329.35 | 47.89 | 3 | 4 | 0 | 4.17 | −5.17 | Moderately | 0 | 0.55 | 1 |
| | | | | | | | | soluble | | | |
| ZINC00709065 | 357.36 | 76.38 | 4 | 4 | 1 | 3.61 | −4.79 | Moderately | 0 | 0.55 | 0 |
| | | | | | | | | soluble | | | |
| ZINC00784494 | 362.4 | 100.44 | 4 | 4 | 1 | 3.83 | −5.05 | Moderately | 0 | 0.55 | 0 |
| | | | | | | | | soluble | | | |
| ZINC00755112 | 369.42 | 65.08 | 1 | 4 | 0 | 3.82 | −5.44 | Moderately | 1 | 0.55 | 0 |
| | | | | | | | | soluble | | | |
| ZINC00786361 | 394.47 | 58.2 | 8 | 2 | 2 | 4.4 | −5.66 | Moderately | 1 | 0.55 | 0 |
| | | | | | | | | soluble | | | |
| ZINC00803271 | 350.8 | 51.1 | 4 | 2 | 1 | 3.92 | −4.9 | Moderately | 0 | 0.55 | 0 |
| | | | | | | | | soluble | | | |
| ZINC01353676 | 369.42 | 89.21 | 5 | 5 | 0 | 3.24 | −4.63 | Moderately | 0 | 0.55 | 0 |
| | | | | | | | | soluble | | | |
| ZINC01786707 | 291.26 | 64.96 | 1 | 5 | 0 | 2.84 | −3.77 | Soluble | 0 | 0.85 | 1 |

^{a}MW = molecular weight (g/mol).
^{b}TPSA = Topological polar surface area (Å²).
^{c}Lipinski rule-of-five: number of violations.
^{d}PAINS: Pan Assay Interference Structures alert identify potentially problematic fragments.

TABLE 3
Predicted pharmacokinetic properties of LCN2 inhibitors and compound derivatives.

| | Pharmacokinetic Properties | | | | | | | | |
|---|---|---|---|---|---|---|---|---|---|
| | GI | BBB | Pgp^{a} | CYP1A2 | CYP2C19 | CYP2C9 | CYP2D6 | CYP3A4 | log Kp |
| Molecule ID | absorption | permeant | substrate | inhibitor | inhibitor | inhibitor | inhibitor | inhibitor | (cm/s)^{b} |
| ZINC00829534 | High | Yes | Yes | Yes | Yes | Yes | No | Yes | −5.62 |
| ZINC02472705 | High | Yes | No | Yes | Yes | No | Yes | No | −5.08 |
| ZINC02298240 | High | Yes | No | Yes | Yes | No | No | No | −4.9 |
| ZINC00348949 | High | Yes | No | Yes | Yes | No | No | No | −4.89 |
| ZINC03901817 | High | Yes | No | Yes | Yes | Yes | No | Yes | −6.08 |
| ZINC00640089 | High | Yes | No | Yes | Yes | Yes | Yes | No | −5.68 |
| ZINC02502578 | High | Yes | No | Yes | Yes | Yes | No | No | −5.54 |
| ZINC00230567 | High | No | No | No | Yes | Yes | Yes | Yes | −5.78 |
| ZINC00324361 | High | Yes | No | Yes | Yes | Yes | No | No | −4.92 |
| ZINC00709065 | High | No | No | Yes | Yes | Yes | No | No | −5.72 |
| ZINC00784494 | High | No | No | Yes | Yes | Yes | No | Yes | −5.55 |
| ZINC00755112 | High | Yes | Yes | Yes | Yes | Yes | No | No | −5.38 |
| ZINC00786361 | High | Yes | Yes | No | Yes | Yes | Yes | Yes | −4.91 |
| ZINC00803271 | High | Yes | No | Yes | Yes | Yes | No | Yes | −5.49 |
| ZINC01353676 | High | No | No | Yes | Yes | Yes | No | No | −5.91 |
| ZINC01786707 | High | Yes | No | Yes | Yes | Yes | No | No | −6.06 |

^{a}Pgp: P glycoprotein substrate.
^{b}Log Kp (cm/s): skin permeation

1. A method of reducing progression and metastasis of inflammatory breast cancer (IBC) cells by inhibiting overexpression of lipocalin-2 (LCN2), the method comprising:
(a) obtaining one or more inhibitors that selectively target a lipocalin-2 (LCN2) calyx; and
(b) contacting the IBC cells with at least one of the inhibitors at concentrations effective to inhibit overexpression of LCN2, as compared to levels of LCN2 in IBC cells not contacted with the inhibitors.

2. The method of claim 11, wherein the small molecules are selected from a group consisting of compounds ZINC00784494, ZINC00640089 and combinations thereof.

3. The method of claim 12, wherein the siRNAs are selected from the group consisting of LCN-2-siRNA1, LCN2-siRNA-2, and combinations thereof.

4. The method of claim 1, wherein IBC cell proliferation and viability are reduced.

5. The method of claim, 1 wherein the IBC cells are further contacted with standard chemotherapy.

6. The method of claim 1, wherein the IBC cells are from a source in which neither HER2 nor EGFR are expressed or mutated.

7. The method of claim 12, wherein the siRNAs significantly reduced at least one characteristic of IBC cells selected from the group consisting of proliferation, viability, migration, invasion, and arrested cell cycle progression in the G0/GI to S phase transition.

8. The method of claim 11, wherein the small molecule significantly decreases cell proliferation, cell viability and AKT phosphorylation levels in SUM 149 cells or equivalents thereof.

9. (canceled)

10. The method of claim 1, wherein the one or more inhibitors are selected from the group consisting of small molecules, siRNAs, or a combination thereof.

11. The method of claim 1, wherein the one or more inhibitors comprise a small molecule.

12. The method of claim 1, wherein the one or more inhibitors comprise siRNAs.

13. The method of claim 11, wherein the small molecule is

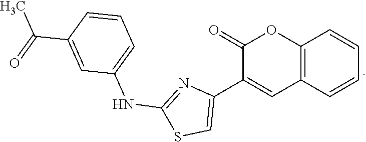 [SMILES: CC(=O)c1cccc(Nc2nc(-c3cc4ccccc4oc3=O)cs2)c1]

14. The method of claim 11, wherein the small molecule is

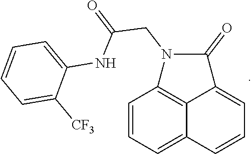 [SMILES: O=C(CN1C(=O)c2cccc3cccc1c23)Nc1ccccc1C(F)(F)F]

15. The method of claim 11, wherein the small molecule is

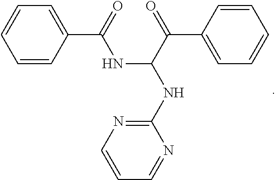 [SMILES: O=C(NC(Nc1ncccn1)C(=O)c1ccccc1)c1ccccc1]

16. The method of claim 11, wherein the small molecule is

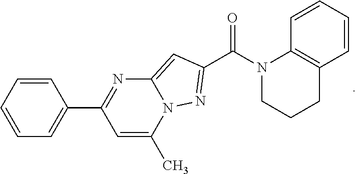 [SMILES: Cc1cc(-c2ccccc2)nc2cc(C(=O)N3CCCc4ccccc43)nn12]

17. The method of claim 8, wherein the small molecule is

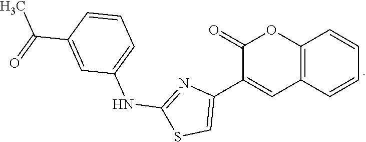 [SMILES: CC(=O)c1cccc(Nc2nc(-c3cc4ccccc4oc3=O)cs2)c1]

18. The method of claim 8, wherein the small molecule is

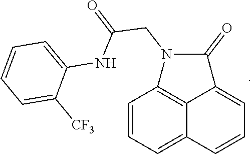 [SMILES: O=C(CN1C(=O)c2cccc3cccc1c23)Nc1ccccc1C(F)(F)F]

19. The method of claim 8, wherein the small molecule is

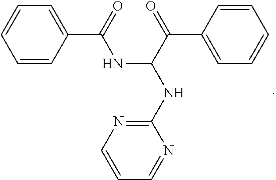 [SMILES: O=C(NC(Nc1ncccn1)C(=O)c1ccccc1)c1ccccc1]

20. The method of claim 8, wherein the small molecule is 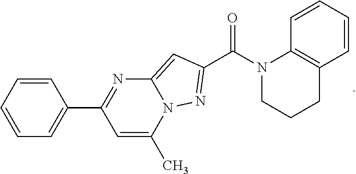 [SMILES: Cc1cc(-c2ccccc2)nc2cc(C(=O)N3CCCc4ccccc43)nn12]
